(19) United States
(12) Patent Application Publication
(10) Pub. No.: US 2004/0030325 A1
(43) Pub. Date: Feb. 12, 2004

(54) Removable attachments for laser emitting devices

(76) Inventors: Nicholas Cahir, Cambridge (GB); Michelle Pataki, Elsternwick (AU)

Correspondence Address: IRELL & MANELLA LLP, 1800 AVENUE OF THE STARS, SUITE 900, LOS ANGELES, CA, 90067, US

(21) Appl. No.: 10/306,776

(22) Filed: Nov. 26, 2002

(30) Foreign Application Priority Data
Dec. 5, 2001 (AU) PR9322
Dec. 5, 2001 (AU) PR9323

(51) Int. Cl. A61B 18/20

(52) U.S. Cl. 606/9; 606/13

(57) ABSTRACT

An attachment for a laser emitting device includes a body having a first end configured to detachably engage a laser emitting device and a second end, and a cap attached to the second end of the body and having a surface configured to contact the skin of a patient. A control device attached to the body is configured to control the operation of the laser emitting device when the first end of the body is engaged with the laser emitting device.

Pursuant to 35 U.S.C. §119, this application claims the priority of Australian Provisional Applications Nos. PR9322 and PR9323, both filed on Dec. 5, 2001, and both of which are incorporated herein in their entirety by reference thereto.

### BACKGROUND OF THE INVENTION

I. Field of the Invention

This invention relates to medical devices, and in particular to laser emitting devices for altering skin permeability.

II. Background of the Invention

The traditional method for the collection of small quantities of fluids, gases or other biomolecules from a patient utilizes mechanical perforation of the skin with a sharp device such as a metal lancet or needle. Additionally, the typical method of administering anesthetics or other pharmaceuticals is through the use of a needle.

These procedures present risks such as the possible infection of health care workers and the public by the sharp device used to perforate the skin. When skin is perforated with a sharp device such as a metal lancet or needle, biological waste is created in the form of the “sharp” contaminated by the patient's blood and/or tissue. If the patient is infected with blood-born agents, such as human immunodeficiency virus (HIV), hepatitis virus, or the etiological agent of any other diseases, the contaminated sharp poses a serious threat to others that might come in contact with it. For example, many medical workers have contracted HIV as a result of accidental contact with a contaminated sharp.

An additional drawback is the cost and difficulty associated with handling and disposal of biologically hazardous waste. Post-use disposal of contaminated sharps imposes both logistical and financial burdens on the end user. These costs are imposed as a result of the social consequences of improper disposal. For example, in the 1980's improperly disposed biological wastes washed up on public beaches on numerous occasions. Improper disposal also permits others, such as intravenous drug users, to obtain contaminated needles and spread disease.

Furthermore, the pain associated with being stabbed by a sharp instrument can be traumatizing, especially for pediatric patients, causing significant stress and anxiety in the patient. This problem is often exacerbated when drawing fluids, gases or other biomolecules, which often require repeated stabbing procedures before sufficient fluid is obtained.

Technologies for applying local anesthetic without the use of needles typically include the use of (a) topical lidocaine mixtures, (b) iontophoresis, (c) carriers or vehicles which are compounds that modify the chemical properties of either the stratum corneum or the pharmaceutical, and (d) sonophoresis which involves modifying the barrier function of stratum corneum by ultrasound. A cream containing lidocaine is commonly used, especially in pediatric patients. However, the cream must to be applied for up to 60 minutes before it is effective, and anesthesia is produced to a depth of only about 4 mm. The lack of lidocaine penetration is a consequence of the barrier function of the stratum corneum. Inherent problems with iontophoresis include the complexity of the delivery system, cost, and unknown toxicology of prolonged exposure to electrical current. Additionally, the use of carriers or vehicles involves additional compounds which might modify the pharmacokinetics of the pharmaceutical of interest or which might be irritating.

Thus, a need exists for methods and equipment to remove fluids, gases or other biomolecules and/or to administer anesthetics or other pharmaceuticals that do not require a sharp instrument to breach the stratum corneum of the patient. Laser emitting devices have been used in recent years as efficient and precise tools in a variety of surgical procedures. Among potentially new sources of laser radiation, the rare-earth elements are of major interest for medicine. One of the most promising of these is a YAG (yttrium, aluminum, garnet) crystal doped with erbium (Er) ions. With the use of this crystal, it is possible to build an erbium-YAG (Er:YAG) laser that can be configured to emit electromagnetic energy at a wavelength (2.94 microns). Radiant energy at this wavelength is strongly absorbed by water and thus tissue, which consists mostly of water. If the intensity of the radiation is sufficient, rapid heating can result followed by vaporization of tissue. In addition, deposition of this energy can result in photomechanical disruption of tissue.

Portable laser emitting devices that address the needs outlined above are disclosed in co-owned U.S. Pat. Nos. 5,643,252, 6,056,738, 6,251,100, 6,315,772, 6,419,642 and 6,425,873, all of which are incorporated herein in their entirety by reference thereto. To function optimally, these and other laser emitting devices should be placed in contact with the patient's skin at the site of treatment (i.e. the site to be irradiated with the laser beam or beams). Thus, the region of the laser emitting device that makes contact with the patient's skin is exposed to patient skin and contaminants thereon. Additionally, during treatment debris is produced in the form of particles of the stratum corneum that have become separated due to the action of the laser, and/or interstitial fluids, gases, and other biomolecules released through the treated skin. Such debris will typically be deposited at least in part on the laser emitting device. However, it is considered generally desirable to be able to use such laser emitting devices on a plurality of patients without having to sterilize the device after each use in order to prevent cross-contamination between patients. The method and devices disclosed herein fulfill this and other needs.

### SUMMARY OF THE INVENTION

In one aspect, an attachment is provided for a laser emitting device, comprising a body having a first end configured to detachably engage a laser emitting device and further having a second end; a cap attached to the second end of the body and having a surface configured to contact the skin of a patient; and a control device attached to the body and configured to control the operation of the laser emitting device when the first end of the body is engaged with the laser emitting device.

In another aspect, an attachment is provided for a laser emitting device, comprising a body having a first end configured to detachably engage a laser emitting device, a second end, and a passage extending from the first end to the second end to pass a laser beam emitted by the laser emitting device therethrough; a cap attached to the second end of the body, the cap having a surface configured to contact the skin of a patient and formed with an opening configured to cooperate with the body passage to pass the laser beam therethrough and onto the skin of the patient; and a control device attached to the body and configured to control the operation of the laser emitting device when the first end of the body is engaged with the laser emitting device.

The control device may be an electric circuit, which may be configured to allow a laser element located in the laser emitting device to draw an electric charge from a power source located in the laser emitting device when the first end of the body is engaged with the laser emitting device. The electric circuit may further contain a fuse element, which may be configured to cease operating after a predetermined amount of electric charge has passed through the electric circuit to prevent the laser element from drawing electric charge from the power source. The electric circuit may also include a memory containing a predetermined code sequence, and may further be configured to communicate the predetermined code sequence to the laser emitting device. Additionally, the electric circuit may be configured to delete the predetermined code sequence from the memory upon operation of the laser emitting device.

In further aspects, the body may be configured to detachably engage the laser emitting device for movement therein when at least a minimum amount of pressure is applied to the cap by contact with the skin to actuate a mechanism in the laser emitting device for operation of the laser emitting device. The cap may be positionable substantially in a focal plane of the laser emitting device when at least the minimum amount of pressure is applied to the cap by contact with the skin. Additionally, a lens of at least semi-transparent material may be positioned between the body and the cap to pass a laser beam emitted by the laser emitting device therethrough.

### BRIEF DESCRIPTION OF THE DRAWINGS

FIG. 1 is a perspective, partially exploded view of an attachment for use with a laser emitting device in accordance with principles of one embodiment described herein;

FIG. 2 is a perspective, partially exploded view of an attachment for use with a laser emitting device in accordance with principles of an alternative embodiment described herein;

FIG. 3 shows front, side, top, and perspective views of an attachment for use with a laser emitting device in accordance with principles of another alternative embodiment described herein;

FIG. 4 shows front, side, top, and perspective views of an attachment for use with a laser emitting device in accordance with principles of yet another alternative embodiment described herein;

FIG. 5 is a partial perspective view of the attachment of FIG. 1 engaged in an initial engagement position with a laser emitting device in accordance with principles of an embodiment described herein;

FIG. 6 is a partial perspective view of the attachment of FIG. 1 engaged in a final engagement position with a laser emitting device in accordance with principles of an embodiment described herein;

FIG. 7 is a partial perspective view of the attachment of FIG. 1 being disengaged from a laser emitting device in accordance with principles of an embodiment described herein;

FIG. 8 is an exploded perspective view of an applicator for attachment to a laser emitting device in accordance with principles of an embodiment described herein;

FIG. 9 is an exploded perspective view of another applicator for attachment to a laser emitting device in accordance with principles of an embodiment described herein; and

FIG. 10 is a perspective view of the applicator shown in FIG. 8 attached to a laser emitting device in accordance with principles of an embodiment described herein.

### DETAILED DESCRIPTION

In one embodiment disclosed herein, a detachable attachment is provided for a laser emitting device to facilitate safe operation of the laser emitting device. More particularly, an attachment is provided for removably attaching to a portable laser emitting device that is configured for irradiating the skin of a patient (such as a human patient) for the purpose of withdrawing fluids or other interstitial or subcutaneous matter from the body of the patient, or for the purpose of introducing a substance into the body of the patient. To this end, an attachment is provided that is formed with engagement means for engaging the laser emitting device and further includes control means to control and permit operation of the laser emitting device.

In one embodiment as described herein, the engagement means may include preliminary and secondary engagement means. The preliminary engagement means may include a first tab located on the distal end of a body portion of the attachment which, on initial engagement of the attachment on the laser emitting device, may engage a pivotal lever of the laser emitting device. The secondary engagement means may include a second tab which may be located on an opposing side of the distal end of the body portion relative to the first tab and which may engage a switch element of the laser emitting device as the attachment is inserted into the laser emitting device to a final operating position so as to activate the laser emitting device.

According to one embodiment described herein, engagement of the attachment with the laser emitting device facilitates activation of the device. Generally, it may be desirable to provide some form of electronic recognition between the attachment and the laser emitting device such that when the attachment is engaged with the device, the device is electronically activated to facilitate operation (i.e. emission of one or more laser beams) of the device. According to one embodiment disclosed herein, the body portion may include electronic identification means such as an electronic circuit (or “chip”) that may cooperate with a sensor of the laser emitting device to control and permit operation of the laser emitting device. To this end, the chip may include electronic identification means readable by the laser emitting device on engagement and insertion of the attachment into the device to the final operating position.

Because debris is produced by the laser beam as it irradiates a patient's skin and because this debris may collect on the attachment, it may be preferable that the attachment be configured as a “single use” attachment. For instance, the attachment may be altered upon cessation of operation of the laser emitting device or removal of the attachment from the device so that it may not be reused with the device. Such single use configuration of the attachment may be achieved by any practicable means. For example, the attachment may be mechanically altered on removal from the laser emitting device so that it cannot be re-engaged with the device. In another embodiment, one or more electronic identification means in the attachment may be adapted to be altered on operation of the device (e.g. by the application of reverse voltage) such that the electronic identification means no longer cooperate with the sensor and thereby prevent further operation of the device. The attachment may also be encoded to ensure single use of the attachment. That is, on engagement with the ablative device the attachment may communicate and effectively “register” with the device so that any subsequent engagement of the particular tip will not activate the device.

The attachment may be formed of a body portion and a contact portion, which may be integral with each other. Alternatively, the body portion may be adapted to engage the contact portion. The body portion and the contact portion may each include a passage configured to cooperate with one another so as to enable operation of a laser of the laser emitting device to emit a laser beam through the attachment and onto the skin of a patient. A lens may be positioned between the passage of the body portion and the passage of the contact portion to prevent debris and other contaminants from entering the attachment and the interior of the laser emitting device itself. The lens may take any suitable form and may be made from any suitable material that is sufficiently transparent to the light of the laser beam or beams emitted by the laser emitting device. In one embodiment, the lens may have a layered structure. In another embodiment, the lens may be formed from teflon.

In one method of use of a laser emitting device, the laser beam may be directed to a very specific site on the patient's skin. In this regard, it may be desirable that the laser beam emitted by the laser emitting device be well defined without any substantial halo effect. To achieve this result, the attachment may be formed with an aperture configured so that the laser beam of the laser emitting device is clipped as it passes through the aperture. As will be understood by those skilled in the art, the term “clipped” refers to the blocking of a portion or portions of the laser beam to ensure that the beam that is emitted from the attachment, and therefore from the device, onto the target area on the skin, is well defined.

In an embodiment as described herein, the body portion includes at its proximal end a flange that is received by a contact portion of the attachment. The flange may be retained within the contact portion by any practicable means. For example, the flanges may be adhered or welded within the contact portion, such as by ultrasonic welding.

It may be desirable in some instances to provide a mark on the skin to clearly indicate the target site for the laser beam. This may be so as to ensure that any further treatment is carried out on the same site, or to clearly indicate the area of irradiation for medicament administration or interstitial fluid collection. In this regard, a proximal end of the body portion of the attachment may include a recess to house an ink pad, wherein the ink pad may be formed with an aperture that is aligned with apertures in the body portion and in the contact portion. The ink pad may further include a plurality of projections that project through a plurality of complimentary apertures in the contact portion when the body portion is engaged by the contact portion. It will be appreciated that the marking material need not be limited to ink, but may be any material that can apply a mark to the skin. Furthermore, any other suitable marking means may be provided to facilitate marking of the skin as desired, the concept disclosed herein being the combination of any such marking means with an attachment as described elsewhere herein.

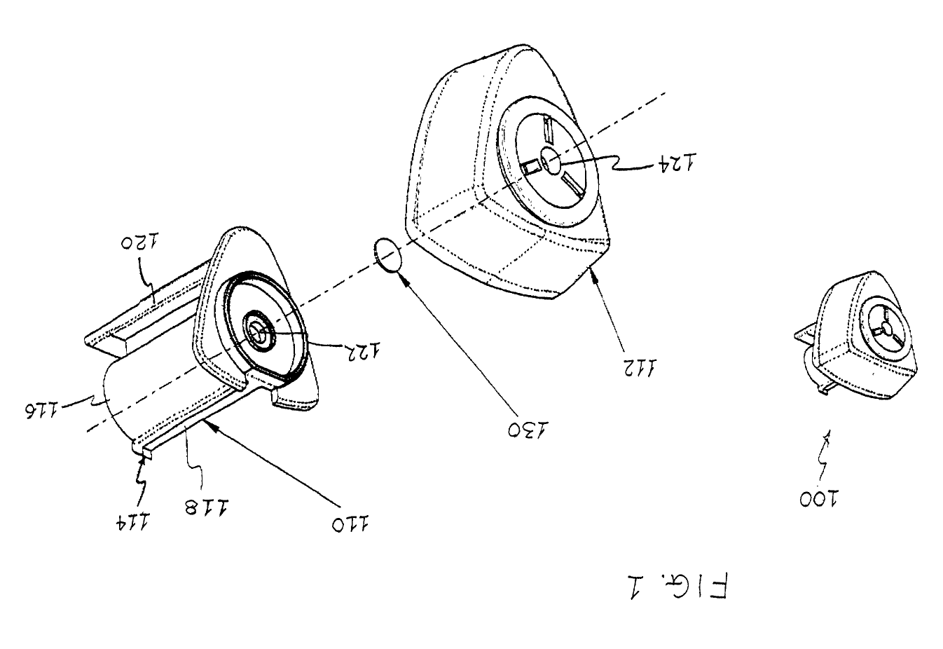

With greater particularity and reference to FIG. 1, one embodiment of an attachment 100 as disclosed herein is illustrated in its assembled form and in a partially exploded view. The attachment 100 includes a body portion 110 and a contact portion 112 that engage each other to form the attachment. As mentioned above, these portions may be formed integral with one another.

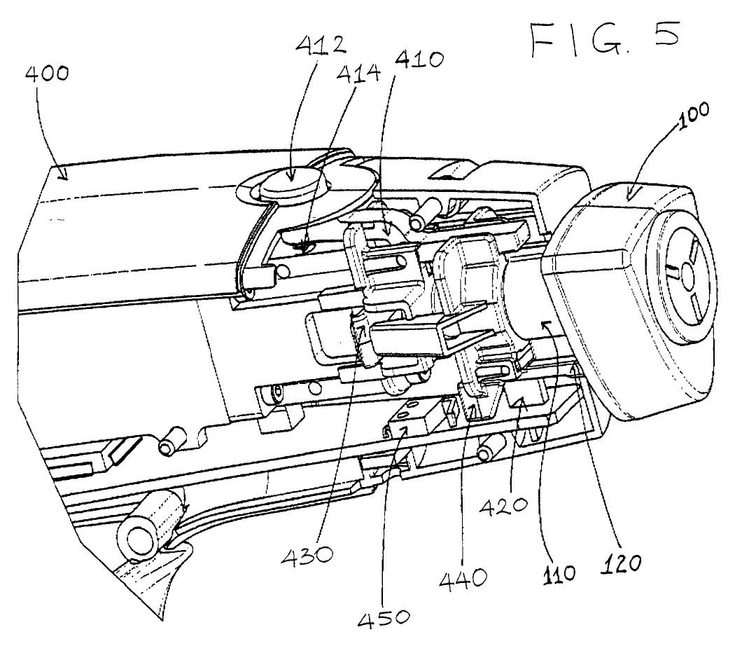

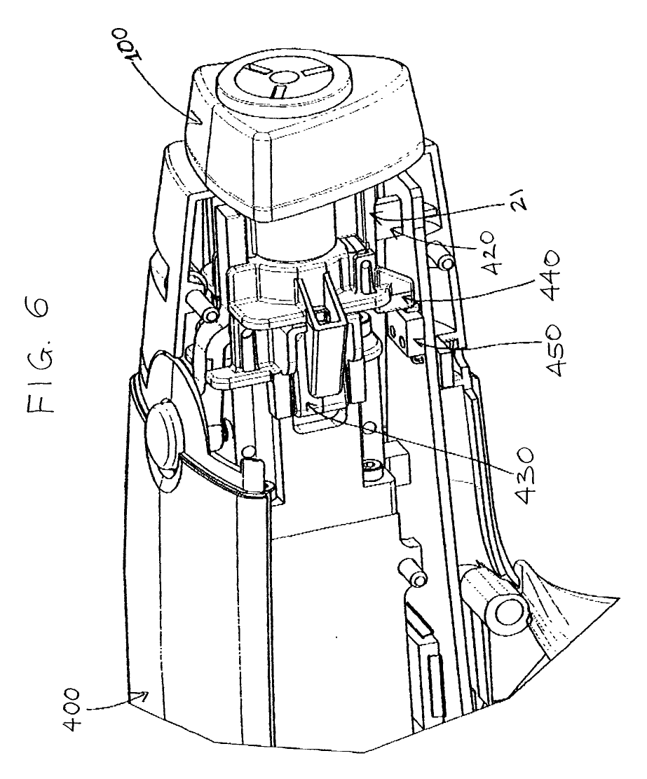

The body portion 110 may include a first tab 114 located on a distal end 116 of the body portion. As disclosed elsewhere herein and best illustrated in FIG. 5, the first tab 114 may be provided to engage a laser emitting device to facilitate engagement of the attachment 100 with the laser emitting device. In one embodiment, the first tab 114 may be located on a longitudinally extending ridge 118 of the body portion 110. The body portion 110 may further include a second tab 120 that may be located on an opposing side of the body portion relative to the first tab 114. The second tab 120 may be extend longitudinally along the body portion 110 and may be adapted to engage the laser emitting device to activate the device (as best illustrated in FIG. 6 and described in detail elsewhere herein).

The body portion 110 may further include an aperture 122 aligned with an aperture 124 formed in the contact portion 112. A lens 130 may also be provided to be held between the body portion 110 and the contact portion 112 in alignment with the aperture 122 of the body portion and the aperture 124 of the contact portion. The aperture 122 of the body portion 110 as well as the aperture 124 of the contact portion 112 may each be sized so as to facilitate clipping of the laser beam as it is emitted through the attachment 100. Generally, the contact portion 112 may be configured to provide a comfortable and effective surface to contact the skin of the patient. Also, the attachment 100 is advantageously designed to allow the passage of at least about 80%, preferably 85%, more preferably about 90%, and most preferably about 92% of the laser burst energy.

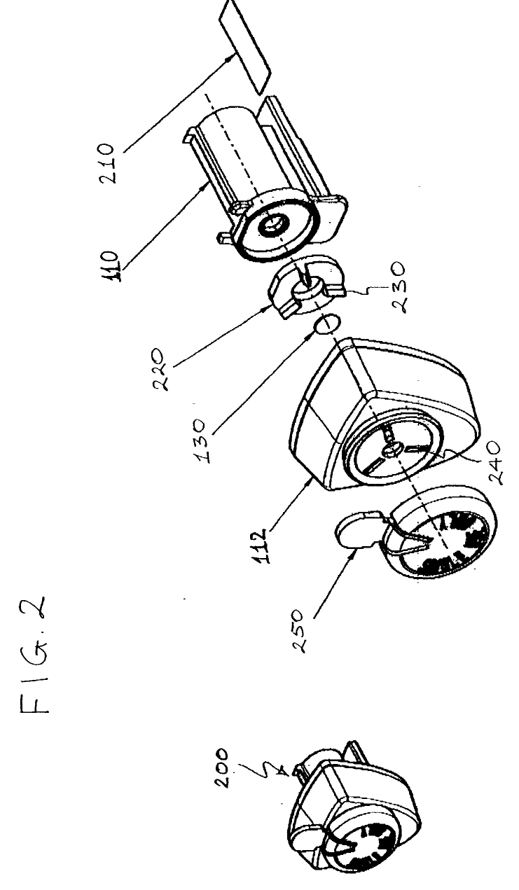

Referring to FIG. 2, in another embodiment as disclosed herein, an attachment 200 may include a control device 210 configured to cooperate with a sensor of a laser emitting device to control and/or enable operation of the laser emitting device. The term “control device” is understood to mean, in the context of the present disclosure, any device capable of performing the functions of interfacing with a laser emitting device for the purpose(s) of controlling and/or enabling/disabling the operation of the laser emitting device. Thus, the control device 210 may be incorporated to ensure that the operation of the laser emitting device is prevented if the attachment 100 is damaged, or it may act as a registration device so that only attachments that include a control device 210 can be used in conjunction with a particular laser emitting device. The control device 210 may further provide a means of ensuring a limited number of uses, typically one use, of the attachment 100. In this regard, as previously mentioned, on operation of the laser emitting device, the control device 210 may be altered to prevent repeated operation of the laser emitting device with the same attachment 200, and thus force the user of the laser emitting device to engage a new, unused attachment 200.

The control device 210 may be a mechanical device, or may be an electronic device incorporating a logic circuit, a memory circuit, and/or a fuse element. A logic circuit may be programmed to communicate with the laser emitting device, such as by providing to the laser device a unique code held in the memory circuit, to thereby enable the laser device to function. The laser emitting device may be provided with its own control logic circuitry that prevents operation of the laser device unless the specific, unique code is received from an attachment 100. After operation of the laser emitting device for a predetermined number of times (typically, one time) the logic circuitry and/or memory may be electronically altered to prevent repeated use of the attachment 100, such as by erasing or modifying the unique code held in the memory circuit.

Alternatively, or additionally, the control device 210 may include a fuse element that may be formed so as to enable operation of the logic circuit and/or the laser emitting device by allowing electric power to flow therethrough to the logic circuit and/or the laser emitting device. The fuse element may be configured to melt and thus break the power circuit to the logic circuit and/or the laser emitting device after a certain amount of electric charge has passed through it. Thus, the fuse element may be configured to melt after an amount of charge has passed through it sufficient to deliver one, two, or any other number of laser radiation doses.

With continued reference to FIG. 2, the attachment 200 may also be provided with an inkpad 220 that may include a plurality of projections 230. The projections 230 may be positioned so that they can protrude through apertures 240 formed in the contact portion 112 when the body portion 110 engages the contact portion 112. In accordance with this embodiment, the attachment 100 may be used to mark the skin of the patient at a desired target area during the laser irradiation process, thereby ensuring that the target site is easily identified on removal of the laser emitting device from the skin. The attachment 200 may further be provided with a cap 250 which may be removable from the contact portion 112 to expose the projections 230 of the inkpad 220 prior to treatment.

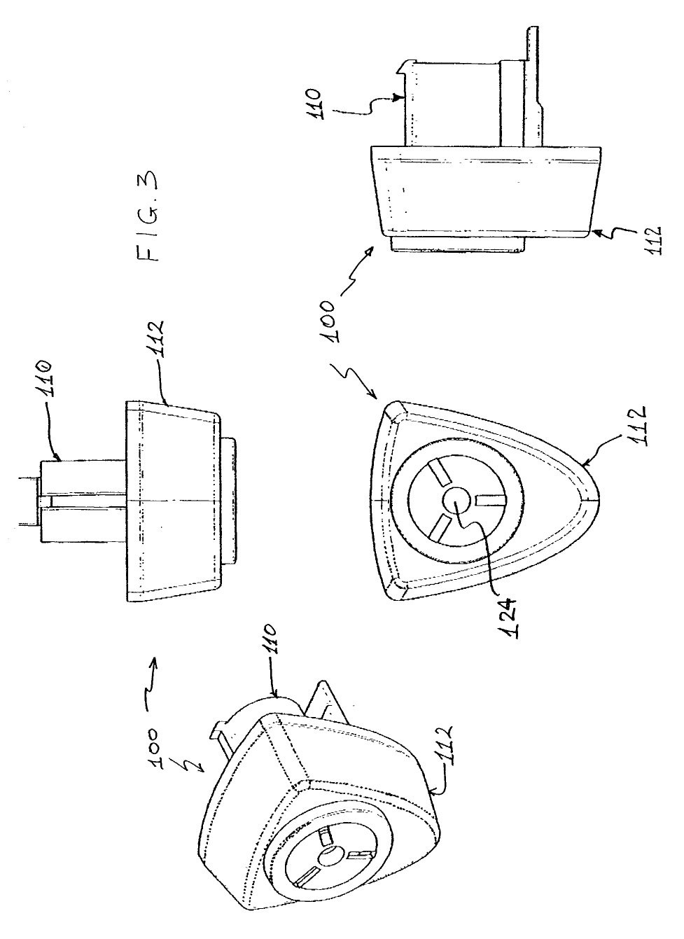

Referring to FIG. 3, in a further embodiment of the attachment 100, the contact portion 112 is formed integrally with the body portion 110. The integral form of the attachment 100 as illustrated may be formed by conventional molding techniques and may include the lens 130, which may be positioned and set during molding of the attachment.

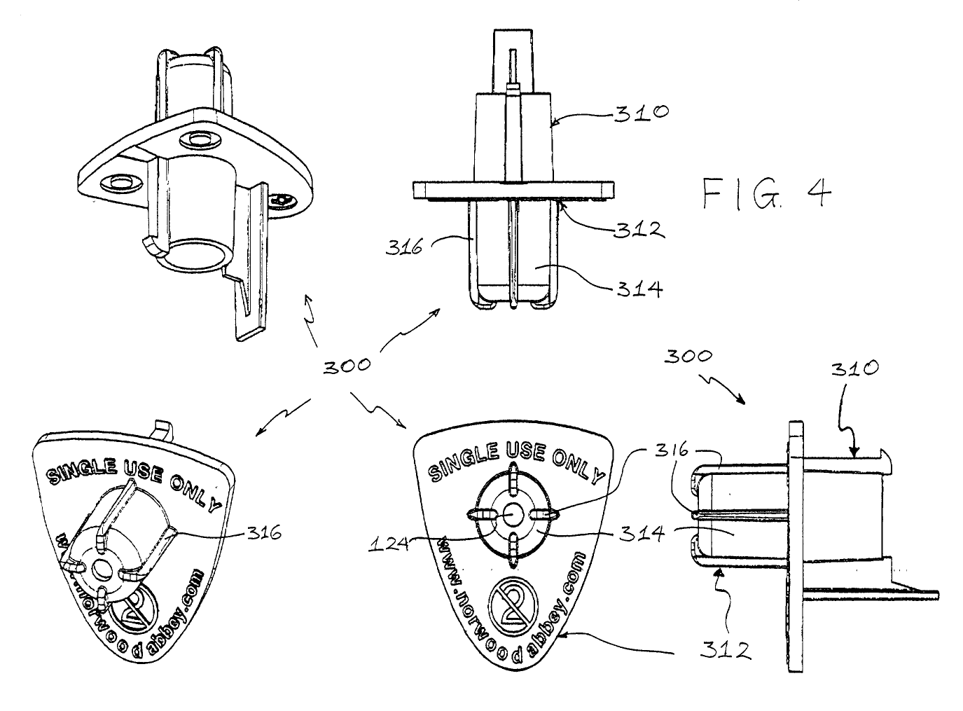

FIG. 4 illustrates another embodiment of an attachment 300 as described herein, formed from a body portion 310 engaged with a contact portion 312. The contact portion 312 of this embodiment is formed with a protruding contact surface 314 extending in alignment with the aperture of the body portion 310 and the aperture of the contact portion and formed with aperture 124 therein. Contact surface 314 may further be formed with longitudinal ridges 316 extending along the length of the contact surface and terminating in the vicinity of contact surface aperture 124 so as to irradiate the skin of a patient when the attachment is brought in contact with the skin. Ridges 316 may thus be contacted with ink or some other marking material prior to treatment so as to mark the target area on the patient's skin when the attachment 100 is brought in contact with the skin.

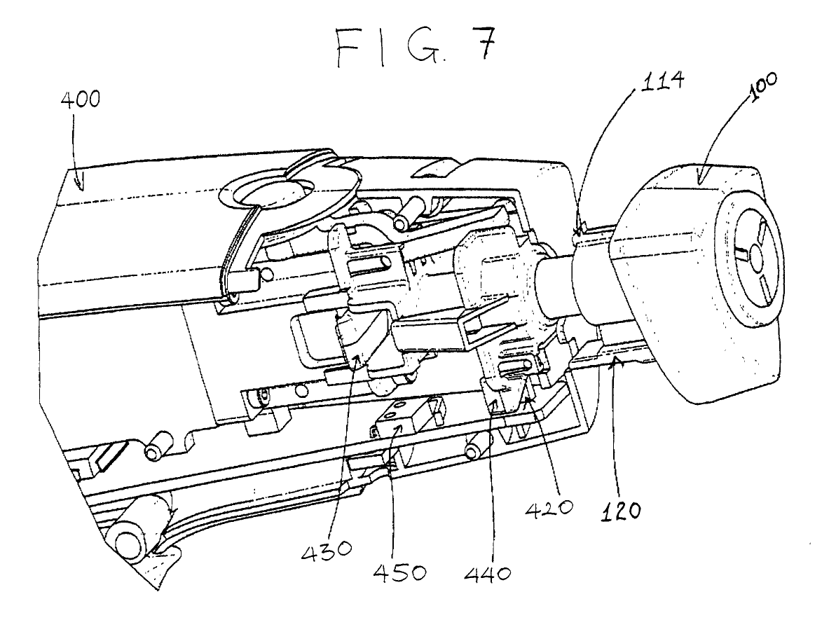

FIGS. 5-7 illustrate one typical method of use of an attachment 100 as disclosed herein with a laser emitting device 400, and more particularly show an initial engagement, a full engagement, and an ejection of the attachment relative to a laser emitting device 400. Referring particularly to FIG. 4, the attachment 100 initially engages the laser emitting device 400 as the first tab 114 of the body portion 110 engages a lever arm 410 of the device 400. The lever arm 410 may include a press button 412 biased by a spring 414. Therefore, as the attachment 100 is forced into the end of the device 400, the first tab 114 engages the lever arm 410 compressing the spring 414 until the first tab 114 is engaged by the lever arm 410. The attachment 100 may thus be configured such that it can only be inserted into the device 400 in one position or orientation. Once inserted in the initial engagement position as illustrated in FIG. 5, the attachment 100 is securely held in the device 400, minimizing the risk of the attachment 100 becoming detached during charging of the laser emitting device 400. During initial engagement, the control device 210 is located in a front portion of the laser emitting device 400. The laser emitting device 400 may include a sensor 420 configured so that it cannot sense or interact with the control device 120 in the initial engagement position.

On further insertion of the attachment 100 into the laser emitting device 400, as illustrated in FIG. 6, the attachment 100 may be moved into a final engagement position. During this movement, the second tab 120 of the attachment 100 may force a shutter 430 provided in the laser emitting device 400 to rotate away from the attachment, while the control device 210 may slide in position to communicate with the sensor 420, thus allowing the sensor 420 access to the control device 210. This attachment method may offer a reliable connection between the sensor and the control device. The rotation of the shutter 430 may allow the second tab 120 to engage an ejector plate 440 that may be configured to move rearward to compress an interlock switch 450. This action and further insertion of the attachment 100 into the device 400 may be used to enable activation of the device 400. Thus, the laser emitting device 400 and the attachment 100 may be configured so that only full insertion of the attachment into the laser device to the position illustrated in FIG. 6 enables operation of the laser device. This is due to the provision of the laser shutter 430 which, when closed, is capable of absorbing a substantial amount of the laser operating wavelength of the laser emitting device 400, preventing any injuries caused by the accidental firing of the laser emitter device. As such, the laser shutter 430 may be formed from any suitable material that will resist the mechanical forces placed upon it by the action of second tab 120 of attachments 100 and that can absorb the required amount of laser energy from the laser beam emitted by the laser emitting device 400.

As best illustrated by FIG. 5, the attachment 100 can be maintained in engaged position while the laser emitting device 400 remains deactivated. It is only upon movement to the second, final position illustrated in FIG. 6 that full engagement of the attachment 100 with the laser emitting device 400 is achieved and operation of the laser device 400 enabled. In accordance with other embodiments described herein, the control device 120 may be configured with an operation time limit that enables operation of the laser emitting device 400 within a predetermined time limit, e.g. 5 seconds, after full engagement of the attachment 100 with the device 400. The time limit may, of course, be selected as desired.

In a method of use as disclosed herein, initial engagement may be achieved by manually placing the attachment 100 onto the tip of the laser emitter device 400, whereas final engagement may be achieved by contacting the skin of the patient with the contact portion and applying sufficient pressure to move the attachment into the final engagement position. In this manner, operation of the laser emitting device 400 will be enabled just before the operator of the laser device is ready to irradiate the patient's skin.

In accordance with another embodiment as disclosed herein, the attachment 100 may be formed so as to align the contact surface of the contact portion 112 in a focal plane of the laser beam emitted by the laser emitting device 400 when the attachment is moved into the final engagement position. In this manner, when the attachment is in the initial engagement position, the focal plane is located within the volume enclosed by the attachment and thus upon an inadvertent emission of a laser beam from the laser device, the focal point of the laser beam (i.e. the point at which the beam is most concentrated and thus can do the greatest amount of damage) will be spaced away from any target such as nearby persons. This feature improves the operational safety of any laser emitting device utilizing an attachment as disclosed herein. This features also enhances the precision of a laser emitting device because it spaces the skin of the patient at the precise focal length of the laser emitting device upon contact of the attachment with the patient's skin, and thus permits the most efficient deposition of the laser beam's energy upon the patient's skin, resulting in reduced power consumption and treatment duration.

Once the operation of the device has been conducted, the attachment 100 may be released and ejected from the laser emitting device 400 by pushing on the exposed button 412 of the lever arm 410. In this manner, removal of the attachment 100 may be achieved without requiring the operator of the laser device to touch the attachment and thereby avoiding possible transfer of contamination from the attachment to the user. On ejection, the shutter 430 may advantageously be returned to its original, closed position (i.e. blocking the laser of the laser device) by a bias spring to block any accidentally discharged laser beams. And also prevent access to the interior of the laser emitting device and thereby excess dust contamination. A laser-transparent membrane may also be provided inside the attachment 100 (such as on the distal end 116 of the body portion 110) to ensure that the interior of the laser emitting device is not contaminated when the attachment is in position on the device. The membrane should be made from a material that is transparent to the laser beam. Examples of such materials include quartz, mica, and sapphire.

In accordance with another embodiment as disclosed herein, the laser emitting device may include one or more LEDs or other indicators that may indicate, among other information, laser charge status, engagement of the attachment with the device, and/or power status of the laser emitting device. The material of construction of the attachment is of no particular importance, but it may be found preferable to manufacture the attachment from a material or materials that are biocompatible and that maximize ease of use while minimizing and risk of adverse reactions, such as allergic reactions.

In accordance with another embodiment as disclosed herein, an applicator is provided for facilitating the delivery of a solution to a treatment surface, and in particular an applicator for use with a laser emitting device for the delivery of a therapeutic solution to the skin of a person or animal. Generally, the applicator may include a receptacle for storage of a solution and that is operable between an open configuration and a closed configuration. The receptacle may further include a contact surface that can sealingly contact the skin of the patient and that includes an aperture in fluid communication with the receptacle when the receptacle is in the open orientation to deliver the solution to the skin. In accordance with a method of use disclosed herein, when the receptacle is translated from the closed configuration to the open configuration, the solution is released from the receptacle to the aperture of the contact surface while the contact surface is maintained in sealing contact with the treatment surface (e.g. skin) so that the solution can contact the skin and be absorbed therethrough. The receptacle may take any suitable form provided that it may be translated from the closed orientation to the open orientation to facilitate delivery of the solution from the receptacle to the aperture to enable the solution to contact the treatment surface.

In one embodiment, the receptacle may include a housing having at least one open partition and an attachable cap, such as a threaded cap, that threadably engages the housing. The cap may include a complimentary partition to engage the partition of the housing when the receptacle is in the closed orientation such that the solution is stored between the partitions. The cap may further be configured to be opened, such as by being rotatable to disengage the partitions of the housing and the cap, translating the receptacle to the open orientation, thereby allowing egress of the solution to the aperture of the contact surface. The housing may include any number of partitions, each of which may constitute a subspace of the receptacle when engaged with the complementary partition of the cap.

In another embodiment as disclosed herein, the receptacle may include a housing having at least one open partition and a cap that engages the housing. The cap may include a complementary partition that engages the partition of the housing when the receptacle is in the closed orientation such that the solution is stored within the receptacle. The cap may further include actuating means to disengage the partitions of the housing and the cap, translating the receptacle to the open orientation, to thereby release the solution to the aperture of the abutting face. The one or more open partitions of the housing may include a pair of opposing tabs that extend from opposing sides of the housing, towards the center of the housing, and the partition of the cap may further include a central tab to engage the tabs of the housing and constitute a gate between the tabs of the housing. The central tab of the cap may be engaged by the actuating means such that upon actuation of the actuating means, the central tab may be disengaged from the tabs of the housing to allow egress of the solution to the aperture of the housing.

The cap and the housing may cooperate with each other in any suitable manner. For instance, the cap may be an integral unit fitted (such as by friction fit) on the housing. The cap may include a body portion hingedly attached to the housing of the receptacle, and an insert portion that includes the central tab and is held between the upper portion of the cap and the housing of the receptacle. The insert portion may include a press button as the actuating means for engaging the central tab.

As previously mentioned, one field of use of the applicators disclosed herein is the delivery of therapeutic and medicament-containing solutions in conjunction with the use of a laser emitting device. To this end, one embodiment of the applicator disclosed herein may be adapted as an attachment to engage a laser emitting device and may further include a window through which the laser beam of the laser device may be emitted. This window and/or an additional window or windows may facilitate viewing of the skin of the patient at the site of treatment. In one embodiment disclosed herein, the applicator may be affixed to the skin surface prior to engagement with the laser emitting device. In this case, the window of the applicator may facilitate viewing of the skin through the applicator to ensure that the applicator is affixed to a desired position on the skin surface. In this manner, it is not necessary for the skin to be viewed through the applicator after operation of the laser emitting device because the area of skin to be treated is automatically targeted due to the positioning of the applicator.

In another embodiment disclosed herein, the window may be located in the cap at a position aligned with the aperture of the housing of the receptacle so that the laser beam emitted by the laser emitting device can pass through the window of the cap and aperture of the housing to irradiate the skin of the patient. The contact surface of the applicator may be provided with an adhesive layer which, on the removal of a protective paper overlaid on the adhesive layer, can enable the applicator to be affixed to the treatment surface.

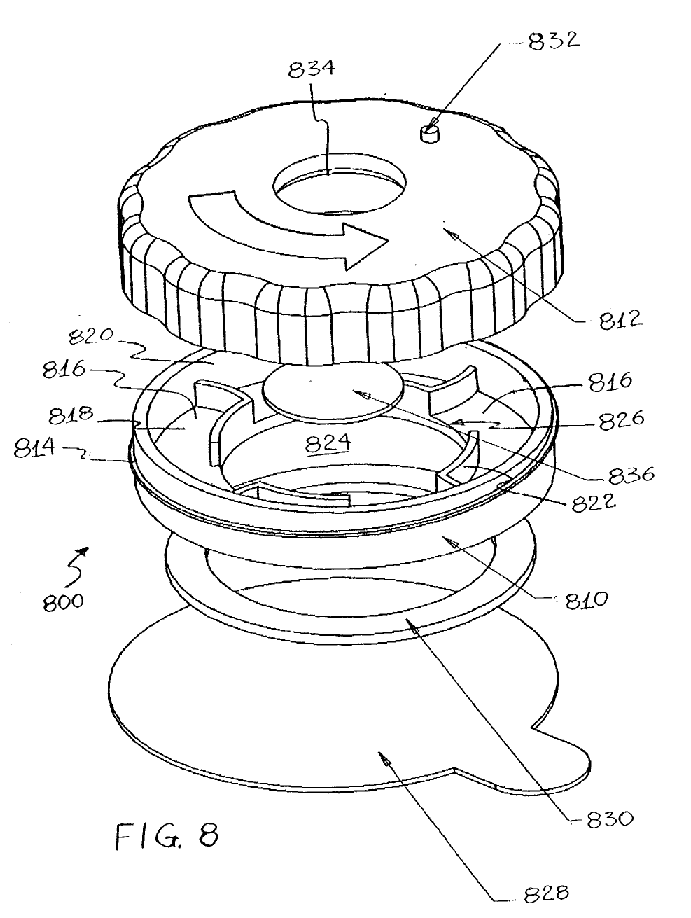

With greater particularity, FIG. 8 illustrates an embodiment of an applicator 800 for use as an attachment with a laser emitting device such as laser emitting device 400 of FIG. 5. The applicator 800 may include a housing 810 and a cap 812. The cap 812 may be threadably mounted on the housing 810 by means of a thread 814 on the housing 810. In the manner described, the housing 810 and cap 812 constitute a receptacle for storage of a solution prior to delivery to a desired surface.

The housing 810 may include a number (four shown for illustration purposes) of partitions 816 spaced about its circumference. The partitions 816 may be configured to cooperate with the outer cap 820 of the housing 810 to define an enclosed volume 818. The inner cap 822 of each partition 816 may be circumferentially positioned about an aperture 824 of the housing 810. The cap 812 may include a number (e.g. four) of complementary partitions (not shown), each engaging an inner cap 822 of a respective partition 816 of the housing 810. In the manner described, when the complementary partitions of the cap 812 engage the partitions 816 of the housing 810, the volume 818 defined by the partitions 816 is enclosed. It is this enclosed volume that stores the solution(s) prior to the delivery of the solution(s) to the desired surface.

The cap 812 may be turned from the closed position, in accordance with the arrow shown on the cap 812, to an open position whereby the complementary partitions of the cap 812 disengage the inner walls 822 of respective partitions 816 of the housing 810. This facilitates the egress of solution(s) held in the spaces 818 through gaps 826 between the inner walls 822 of the partitions 816 to the aperture 824.

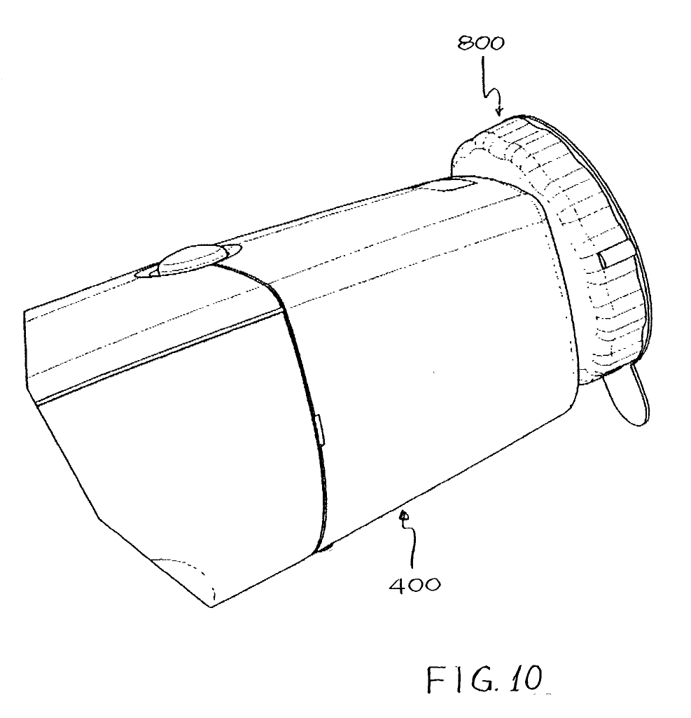

In use, the applicator 800 may be attached to the treatment surface (e.g. the skin of a person to whom a medicament is to be administered) by removing a protective layer 828 from the applicator 800 to uncover an adhesive layer 830 located on the underside of the housing 810. Once the applicator 800 is secured to the surface of the skin, a laser emitting device such as laser emitting device 400 (shown in FIGS. 5 and 10) may be engaged with the cap 812 of the applicator 800. Engagement of the laser emitting device with the applicator 800 may be confirmed by an engagement pin 832 that may be configured to interact with the laser emitting device.

Having engaged the applicator 800, the laser emitting device may next be operated to emit one or more laser beams through an aperture 834 formed in the cap 812 and housing a lens 836. The laser beam(s) emitted through the aperture 834 of the cap 812 will then proceed through the lens 836 and the aperture 824 of the housing 810 to irradiate the patient's skin. Once a desired amount of irradiation has been deposited, operation of the laser emitting device may cease. The cap 812 may then be rotated to an open configuration to release the solution containing one or more desired medicaments from the spaces 818 of the housing 810, following which the solution(s) egress to the aperture 824 to contact the treated skin surface. At this time the applicator 800 can be held in place by the adhesive layer 830 as desired and for a time period sufficient to allow permeation of the solution through the irradiated skin surface.

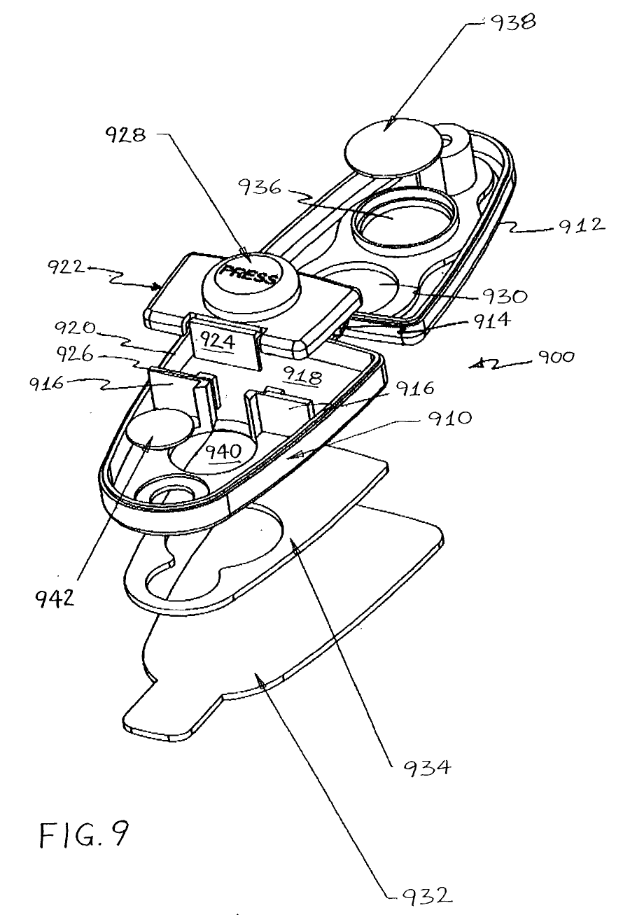

Referring to FIG. 9, another embodiment of an applicator 900 disclosed herein also may include a housing 910 and a cap 912. The cap 912 in this instance may be hingedly attached to the housing 910 by means of a hinge 914. The housing 910 may include partitions 916 that define an enclosed volume 918 with the outer cap 920 of the housing 910. The volume 918 is provided to store the solution to be delivered to the surface of the skin prior to use of the applicator 900.

The applicator 900 may be provided with an insert 922 that may be held between the housing 910 and the cap 912. The insert 922 may alternatively be integrally formed in the cap 912. The insert 922 may include a tab 924 that engages the partitions 916 of the housing 910 by sliding into slots 926 of the partitions 916. The tab 924 may be engaged by a press button 928 of the insert 922 that projects through an aperture 930 of the cap 912 when the cap 912 is engaged with the housing 910.

In one method of use of applicator 900, a protective layer 932 may be removed to uncover an adhesive layer 934 located on the underside of the housing 910. The applicator 900 may then be positioned on the targeted area of skin and fixed thereto by the adhesive layer 934. At this time the cap 32 is engaged with the housing 910, the press button 928 protruding through the aperture 930 of the cap 912 and the tab 924 of the insert 922 engaging the partitions 916 of the housing 910. Once the applicator 900 is secured in place on the skin, a laser emitting device may be engaged with the cap 912 and operated to emit one or more laser beams. The laser beam(s) will proceed through an aperture 936 of the cap 912 that houses a lens 938. The aperture 936 and lens 938 are positioned directly above an aperture 940 of the housing 910 so that the laser beam(s) emitted by the laser emitting device can pass through both the aperture 936 of the cap 912 and the aperture 940 of the housing 910 to irradiate the skin surface beneath the aperture 940.

Once a desired degree of irradiation has been delivered, the operation of the laser of the laser emitting device may cease. A solution held within the enclosed volume 918 of the housing 910 may then be released to the aperture 940 by pressing the press button 928 to cause disengagement of the tab 924 from the partitions 916 of the housing 910. The applicator 900 may then be maintained in position over the skin for a desired time period to allow permeation of the solution into the skin. The time frame may be measured by a color change compound which may be included as a tablet or other indicator 942 located in the housing 910.

Referring to FIG. 10, the applicator 800 may be easily attached to a laser emitting device 400 for use as described elsewhere herein. Attachment may be achieved by any suitable means, including those as described previously with respect to attachments 100, 200, and 300.

Having now described the invention in accordance with the requirements of the patent statutes, those skilled in this art will understand how to make changes and modifications to the present invention to meet their specific requirements or conditions. Such changes and modifications may be made without departing from the scope and spirit of the invention as disclosed herein.

What is claimed is:

1. An attachment for a laser emitting device, comprising:
a body having a first end configured to detachably engage a laser emitting device and further having a second end;
a cap attached to the second end of the body and having a surface configured to contact the skin of a patient; and
a control device attached to the body and configured to control the operation of the laser emitting device when the first end of the body is engaged with the laser emitting device.

2. The attachment of claim 1, wherein the control device comprises:
an electric circuit.

3. The attachment of claim 2, wherein the electric circuit comprises:
an electric circuit configured to allow a laser element located in the laser emitting device to draw an electric charge from a power source associated with the laser emitting device when the first end of the body is engaged with the laser emitting device.

4. The attachment of claim 3, wherein the electric circuit comprises:
a fuse element configured to cease operating after a predetermined amount of electric charge has passed through the electric circuit to prevent the laser element from drawing electric charge from the power source.

5. The attachment of claim 2, wherein the electric circuit comprises:
a memory containing a predetermined code sequence.

6. The attachment of claim 5, wherein the electric circuit comprises:
an electric circuit configured to communicate the predetermined code sequence to the laser emitting device.

7. The attachment of claim 5, wherein the electric circuit comprises:
an electric circuit configured to delete the predetermined code sequence from the memory upon operation of the laser emitting device.

8. The attachment of claims 1 or 2, wherein the body comprises:
a body configured to detachably engage the laser emitting device for movement therein when at least a minimum amount of pressure is applied to the cap by contact with the skin to actuate a mechanism in the laser emitting device for operation of the laser emitting device.

9. The attachment of claim 8, wherein the cap is positionable substantially in a focal plane of the laser emitting device when at least the minimum amount of pressure is applied to the cap by contact with the skin.

10. The attachment of claim 9, further comprising:
a lens of at least semi-transparent material positioned between the body and the cap to pass a laser beam emitted by the laser emitting device therethrough.

11. The attachment of claims 1 or 2, further comprising:
a lens of at least semi-transparent material positioned between the body and the cap to pass a laser beam emitted by the laser emitting device therethrough.

12. The attachment of claim 11, wherein the body comprises:
a body configured to detachably engage the laser emitting device for movement therein when at least a minimum amount of pressure is applied to the cap by contact with the skin to actuate a mechanism in the laser emitting device for operation of the laser emitting device.

13. The attachment of claim 12, wherein the cap is positionable substantially in a focal plane of the laser emitting device when at least the minimum amount of pressure is applied to the cap by contact with the skin.

14. An attachment for a laser emitting device, comprising:
a body having a first end configured to detachably engage a laser emitting device, a second end, and a passage extending from the first end to the second end to pass a laser beam emitted by the laser emitting device therethrough;
a cap attached to the second end of the body, the cap having a surface configured to contact the skin of a patient and formed with an opening configured to cooperate with the body passage to pass the laser beam therethrough and onto the skin of the patient; and
a control device attached to the body and configured to control the operation of the laser emitting device when the first end of the body is engaged with the laser emitting device.

15. The attachment of claim 14, wherein the control device comprises:
an electric circuit.

16. The attachment of claim 15, wherein the electric circuit comprises:
an electric circuit configured to allow a laser element located in the laser emitting device to draw an electric charge from a power source associated with the laser emitting device when the first end of the body is engaged with the laser emitting device.

17. The attachment of claim 16, wherein the electric circuit comprises:
a fuse element configured to cease operating after a predetermined amount of electric charge has passed through the electric circuit to prevent the laser element from drawing electric charge from the power source.

18. The attachment of claim 15, wherein the electric circuit comprises:
a memory containing a predetermined code sequence.

19. The attachment of claim 18, wherein the electric circuit comprises:
an electric circuit configured to communicate the predetermined code sequence to the laser emitting device.

20. The attachment of claim 18, wherein the electric circuit comprises:
an electric circuit configured to delete the predetermined code sequence from the memory upon operation of the laser emitting device.

21. The attachment of claims 14 or 15, wherein the body comprises:
a body configured to detachably engage the laser emitting device for movement therein when at least a minimum amount of pressure is applied to the cap by contact with the skin to actuate a mechanism in the laser emitting device for operation of the laser emitting device.

22. The attachment of claim 21, wherein the cap is positionable substantially in a focal plane of the laser emitting device when at least the minimum amount of pressure is applied to the cap by contact with the skin.

23. The attachment of claim 22, further comprising:
a lens of at least semi-transparent material positioned between the body and the cap to pass a laser beam emitted by the laser emitting device therethrough.

24. The attachment of claims 14 or 15, further comprising:
a lens of at least semi-transparent material positioned between the body and the cap to pass a laser beam emitted by the laser emitting device therethrough.

25. The attachment of claim 24, wherein the body comprises:
a body configured to detachably engage the laser emitting device for movement therein when at least a minimum amount of pressure is applied to the cap by contact with the skin to actuate a mechanism in the laser emitting device for operation of the laser emitting device.

26. The attachment of claim 25, wherein the cap is positionable substantially in a focal plane of the laser emitting device when at least the minimum amount of pressure is applied to the cap by contact with the skin.

27. An attachment for a laser emitting device, comprising:
engagement means configured to detachably engage a laser emitting device;
a contact surface attached to the engagement means and configured to contact the skin of a patient; and
control means attached to the body and configured to control the operation of the laser emitting device when the attachment is engaged with the laser emitting device.

28. The attachment of claim 27, wherein the control means comprise:
an electric circuit.

29. The attachment of claim 28, wherein the electric circuit comprises:
an electric circuit configured to allow a laser element located in the laser emitting device to draw an electric charge from a power source associated with the laser emitting device when the attachment is engaged with the laser emitting device.

30. The attachment of claim 29, wherein the electric circuit comprises:
a fuse element configured to cease operating after a predetermined amount of electric charge has passed through the electric circuit to prevent the laser element from drawing electric charge from the power source.

31. The attachment of claim 28, wherein the electric circuit comprises:
a memory containing a predetermined code sequence.

32. The attachment of claim 31, wherein the electric circuit comprises:
an electric circuit configured to communicate the predetermined code sequence to the laser emitting device.

33. The attachment of claim 31, wherein the electric circuit comprises:
an electric circuit configured to delete the predetermined code sequence from the memory upon operation of the laser emitting device.

34. The attachment of claims 27 or 28, wherein the engagement means comprise:
a body configured to detachably engage the laser emitting device for movement therein when at least a minimum amount of pressure is applied to the cap by contact with the skin to actuate a mechanism in the laser emitting device for operation of the laser emitting device.

35. The attachment of claim 34, wherein the contact surface is positionable substantially in a focal plane of the laser emitting device when at least the minimum amount of pressure is applied to the contact surface by contact with the skin.

36. The attachment of claim 35, further comprising:
means formed from at least semi-transparent material and associated with the contact surface to pass a laser beam emitted by the laser emitting device therethrough.

37. The attachment of claims 27 or 28, further comprising:
means formed from at least semi-transparent material and associated with the contact surface to pass a laser beam emitted by the laser emitting device therethrough.

38. The attachment of claim 37, wherein the engagement means comprise:
a body configured to detachably engage the laser emitting device for movement therein when at least a minimum amount of pressure is applied to the contact surface by contact with the skin to actuate a mechanism in the laser emitting device for operation of the laser emitting device.

39. The attachment of claim 38, wherein the contact surface is positionable substantially in a focal plane of the laser emitting device when at least the minimum amount of pressure is applied to the contact surface by contact with the skin.
